(19) United States
(12) Patent Application Publication
(10) Pub. No.: US 2023/0165610 A1
(43) Pub. Date: Jun. 1, 2023

(54) BONE FIXATION ASSEMBLY

(71) Applicant: MEDARTIS AG, Basel (CH)

(72) Inventors: Ryan SCHLOTTERBACK, Warsaw, IN (US); John R. PEPPER, Warsaw, IN (US)

(73) Assignee: MEDARTIS AG, Basel (CH)

(21) Appl. No.: 18/060,069

(22) Filed: Nov. 30, 2022

Related U.S. Application Data
(60) Provisional application No. 63/266,410, filed on Jan. 5, 2022.
(60) Provisional application No. 63/264,728, filed on Dec. 1, 2021.

(51) Int. Cl. A61B 17/80; A61B 17/74

(52) U.S. Cl. CPC A61B 17/8085; A61B 17/744; A61B 17/8057

(57) ABSTRACT

A bone fixation assembly including a first member, a second member and a link member extending between the first member and the second member. The first and second members each including a slot formed in an axial surface of a shaft extending along a longitudinal axis. The link member including a first lateral side being slidably received in the slot of the first member and a second lateral side being slidably received in the slot of the second member.

### FIELD OF THE INVENTION

This application relates generally to apparatuses, devices, and methods for bone fixation and more particularly to apparatuses, devices, and methods for addressing osteoporotic hip fractures related to skeletal fracture fixation and instrumentation to facilitate fracture reduction.

### BACKGROUND

Hip fractures are a common injury, especially for the elderly having a decrease in bone mass caused by, for example, reduced biosynthetic and replicative potential of osteoblasts, increased osteoclast activity, reduced physical activity, genetic predisposition, decreased calcium intake and hormonal influences. Also, people with osteoporosis often have other medical conditions that lead to an increased rate of falling resulting in a hip fracture.

There are three broad categories of hip fractures based on the location of the fracture: femoral neck fractures, intertrochanteric fractures and subtrochanteric fractures. The femoral neck is the most common location for a hip fracture. The femoral neck is the region of the femur bounded by the femoral head proximally and the greater and lesser trochanters. A femoral neck fracture is intracapsular, e.g., within the hip joint and beneath the fibrous joint capsule.

In general, depending on the type and severity of the fractures, whether classified as stable and unstable, there are numerous operative treatment and management options currently available. Stable fractures are non-displaced fractures that exhibit no deformity or impacted in a valgus positions. Stable femoral neck fractures are generally best treated with surgical stabilization and immediate mobilization.

Typically, treatment of a hip fracture is by operative pinning with two or three parallel cannulated screws 100 placed adjacent to the femoral neck cortex, as illustrated in FIGS. 1A-1C. Pinning is typically chosen because the risks of arthroplasty are high for young patients, and their rate of healing is high due to the absence of osteoporosis. As age and osteoporosis increase, the rate of failure caused by, for example, nonunion, secondary displacement or avascular necrosis, increases. The advantages with pinning include low cost, fast OR time, less invasive surgery, less blood loss, and less postoperative morbidity. However, pinning treatment provides only marginal resistance to subsidence, carries a higher risk of more surgery in the future and does not always adequately resist rotation of bone fragments during healing.

Another operative treatment option is hemi- or total joint arthroplasty. Arthroplasty results in more acute postoperative morbidity, but offer fewer reoperations for nonunion, hardware failure and osteonecrosis. Hemi- or total joint arthroplasty is associated with a lower rate of repeat surgery than internal fixation and is often the better option for older patients. Complications from a hemi-arthroplasty include, for example, dislocation, fracture and infection. The treatment for a failed hemiarthroplasty is conversion to a total hip replacement. Total joint replacement is typically performed on an active patient or one with preexisting arthritis. The failures of total hip replacement are similar to those of a hemi-arthroplasty. The cost and OR time for arthroplasty operations are high.

Other operative treatment options that have been used depending on the stability of the fracture and age and condition of the patient are the use of a blade plate for hip fracture nonunion treatment, CHS/DHS dynamic hip and condylar screw assemblies designed to provide stable internal fixation, sliding hip screw, and intramedullary hip screws for intertrochanteric or subtrochanteric fractures. Failures from these other options include, for example, nonunion, screw cut-out, nail breakage, limp and stress riser in the bone. These other options provide marginal to reasonable resistance to subsidence at higher costs. However, similar to pinning, the use of an intramedullary hip screw does not control rotation of bone fragments during healing.

Thus, there is a need for a device that provides improved outcomes for the treatment of hip fractures at a reasonable cost as compared to existing operative treatment options.

### SUMMARY OF THE INVENTION

Briefly, a bone fixation assembly constructed in accordance with one or more aspects of the present invention provides, for example, an improved device for addressing osteoporotic hip fractures.

One embodiment includes a bone fixation assembly including a first member, a second member and a link member. The first member includes a shaft extending along a longitudinal axis. The shaft includes a proximal end and a distal end. The shaft includes an axial surface extending between the proximal end and the distal end. The shaft includes a slot in the axial surface extending from the proximal end along the longitudinal axis. The second member includes a shaft extending along a longitudinal axis. The shaft includes a proximal end and a distal end. The shaft includes an axial surface extending between the proximal end and the distal end. The shaft includes a slot in the axial surface extending from the proximal end along the longitudinal axis. The link member includes a first lateral side, a second lateral side and a plate member extending between the first lateral side and the second lateral side. The first lateral side is slidably received by the slot in the axial surface of the shaft of the first member. The second lateral side is slidably received by the slot in the axial surface of the shaft of the second member.

In one embodiment, the link member includes a plurality of apertures formed in the plate member. In yet another embodiment, a third member passes through one of the apertures in the plate member.

In yet another embodiment, the plate member of the link member is flexible, and capable of compressing and/or expanding.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

FIGS. 1A-1C depict a prior art treatment option involving the installation of two or more screw members known as pinning;

FIG. 2A-2B depict one example of a bone fixation assembly constructed in accordance with one or more aspects of the present invention being installed into a hip joint;

FIG. 3A depicts a perspective of one example of a screw member used in the bone fixation assembly constructed in accordance with one or more aspects of the present invention;

FIG. 3B depicts proximal end and two side views of one example of screw members used in the bone fixation assembly constructed in accordance with one or more aspects of the present invention

FIG. 4 depicts another example of a bone fixation assembly constructed in accordance with one or more aspects of the present invention being installed into a hip joint;

FIG. 5 depicts a cross sectional view a bone fixation assembly constructed in accordance with one or more aspects of the present invention being installed into a hip joint;

FIG. 6 depicts another example of a bone fixation assembly constructed in accordance with one or more aspects of the present invention being installed in a vertical orientation into a hip joint;

FIG. 7 depicts another example of a bone fixation assembly constructed in accordance with one or more aspects of the present invention being installed into a hip joint with a bone plate;

FIG. 8 depicts another example of a bone fixation assembly constructed in accordance with one or more aspects of the present invention being installed into a hip joint with a bone plate including barrels or sleeves extending from the backside of the bone plate;

FIG. 9 depicts another example of a bone fixation assembly constructed in accordance with one or more aspects of the present invention being installed into a hip joint with an IM nail;

FIG. 10A depicts another example of a link member that may be used with a bone fixation assembly constructed in accordance with one or more aspects of the present invention;

FIG. 10B depicts the link member illustrated in FIG. 10A with a third screw member passing through an opening formed therein;

FIG. 10C depicts the link member and the screw member illustrated in FIGS. 10A and 10B implanted in a calcaneous bone;

FIG. 11A depicts another example of a link member that may be used with a bone fixation assembly constructed in accordance with one or more aspects of the present invention;

FIG. 11B depicts the link member illustrated in FIG. 11A in a compressed state;

FIG. 11C depicts the link member illustrated in FIG. 11A linking two screw members pursuant to one or more aspects of the present invention;

FIG. 12 depicts an alternative embodiment of a bone fixation assembly constructed in accordance with one or more aspects of the present invention illustrating the use of fully threaded screw members;

FIG. 13A depicts an alternative embodiment of a bone fixation assembly constructed in accordance with one or more aspects of the present invention illustrating the use of trailing thread screw members; and

FIG. 13B depicts the bone fixation assembly illustrated in FIG. 13A implanted in a calcaneous bone.

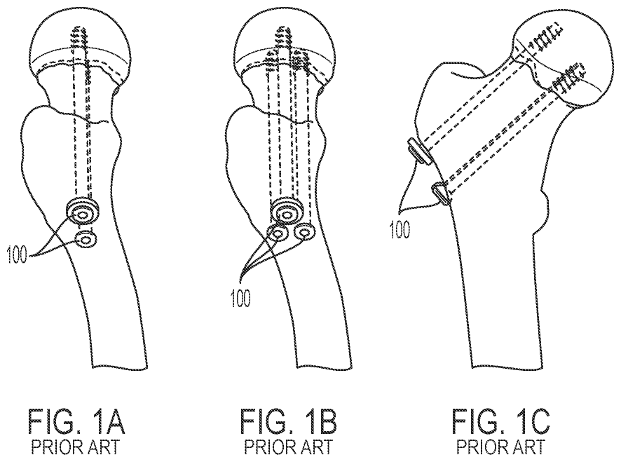

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention. Thus, all the implementations described below are exemplary implementations provided to enable persons skilled in the art to make or use the embodiments of the disclosure and are not intended to limit the scope of the disclosure, which is defined by the claims.

The following description references assemblies, methods, and apparatuses for use to address osteoporotic hip fractures. However, those possessing an ordinary level of skill in the relevant art will appreciate that fixation of other bones, including, for example, the humerus bone, are suitable for use with the foregoing assemblies, methods and apparatuses. Likewise, the various figures, steps, procedures and work-flows are presented only as an example and in no way limit the assemblies, methods or apparatuses described to performing their respective tasks or outcomes in different time-frames or orders. The teachings of the present invention may be applied to fixation related to any bone.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

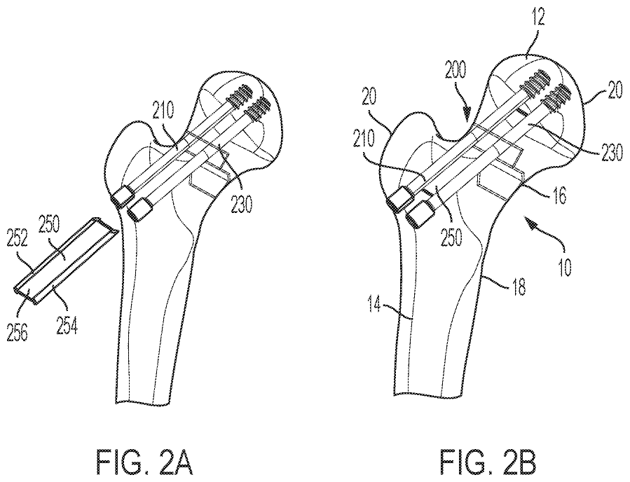

FIGS. 2A-2B depict one example of bone fixation assembly 200 constructed in accordance with one or more aspects of the present invention. In this example, bone fixation assembly 200 is used in a hip joint to fix a hip fracture. The femur 10 includes a proximally round head 12. The proximal femur 10 has a long shaft 14. The femoral neck 16 connects to the femoral head 12 and the long shaft 14. The femur 10 features a hard thin shaft cortex 18 that is thicker than the cortex of the neck 20. The proximal femoral cortex is filled with spongy-like bone (e.g. cancellous bone).

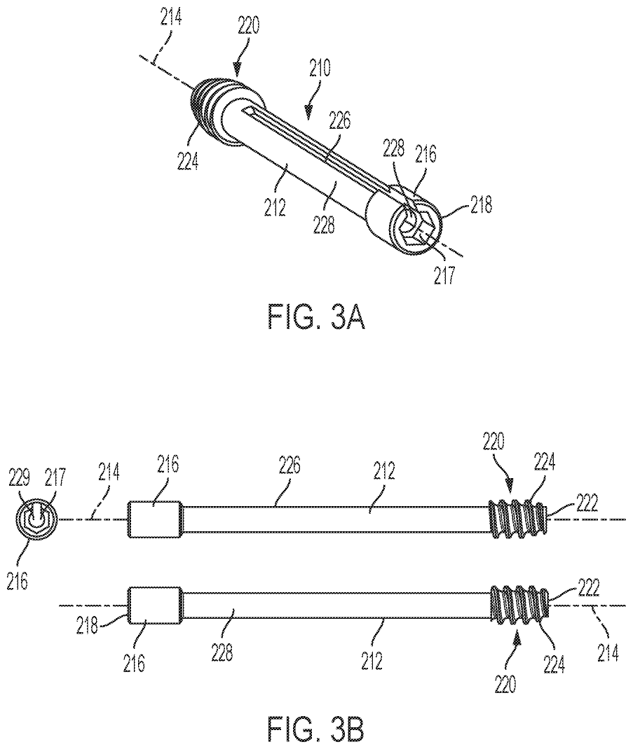

Bone fixation assembly 200 comprises two screw members 210 and 230 and a link member 250 connecting or linking together screw member 210 and screw member 220. As illustrated in FIGS. 3A-3B, one example of screw members 210, 230 may include a cannulated shaft 212 having a longitudinal axis 214, with a head 216, at a proximal end 218, and a distal tip 220, at the distal end 222. Shaft 212 may be cylindrical in shape and comprises threads 224 on, at least, distal tip 220. Head 216 includes an opening 217 configured to receive a driving tool (e.g. screw driver) to assist in insertion of screw members 210, 230 into bone. Screw members 210, 230 may also include a slot 226 formed in an axial side 228 of screw members 210, 230 extending longitudinally along longitudinal axis 214 from proximal end 218 towards distal tip 220. Slot 226 also extends radially inward toward longitudinal axis 214 and connects to a channel 229 extending longitudinally from opening 217 along longitudinal axis 114.

In one example depicted in FIGS. 2A-2B, link member 250 connects or links screw member 210 to screw member 230. Link member 250 comprises a first lateral side 252, a second lateral side 254 and a plate member 256 extending between first lateral side 252 and second lateral side 254. First and second lateral sides 252, 254 may be cylindrical in shape and sized to fit through openings 217, and be slidably received by channels 229, of screw members 210, 230. Plate member 256 may be sized to be slidably received by slots 226 of screw members 210, 230. In one example, as depicted in FIG. 2B, link member 250 extends along the entire length of slots 226 and channels 229 of screw members 210, 230.

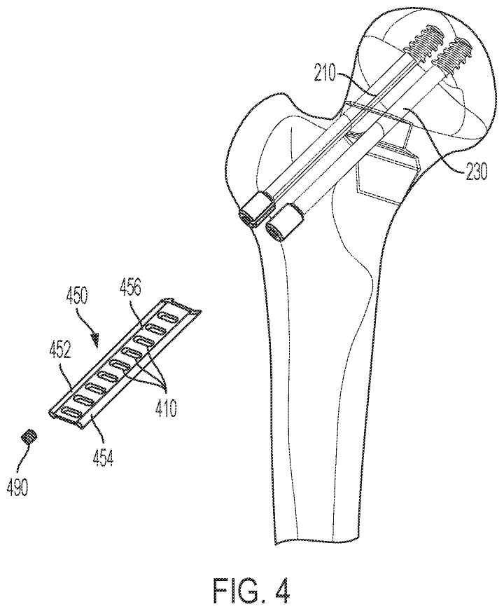

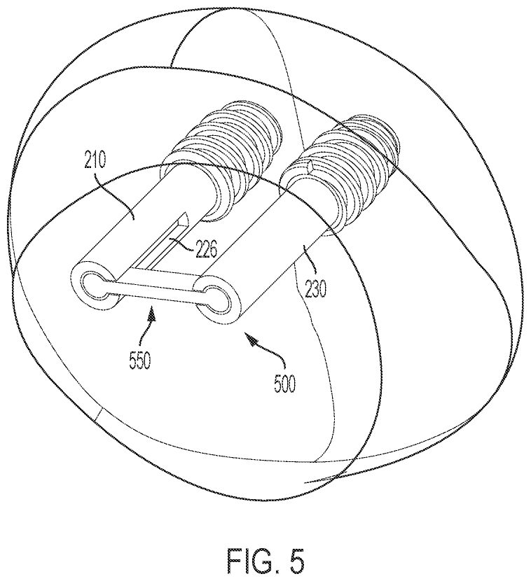

In one example depicted in FIGS. 2A-2B, plate member 256 of link member 250 may be a flat solid plate. In an alternative embodiment depicted in FIG. 4, plate member 456 of link member 450 may include one or more apertures 460 extending between first lateral side 452 and second lateral side 454. As illustrated in FIG. 4, plate member 456 may include a plurality of apertures 460 spaced apart from each other. Apertures 460 may allow for more blood flow providing vascular access as the bone fracture heals. In yet another embodiment depicted in FIG. 5, link member 550 of bone fixation assembly 500 may extend along only a portion of the entire length of slots 226 and channels 229 of screw members 210, 230.

In one example, a locking screw 490, as depicted in FIG. 4, may be used to retain first lateral side 452 and second lateral side 454 of link member 450 within channels 229 of first and second screw members 210, 230.

A bone fixation assemblies constructed in accordance with one or more aspects of the present invention that, for example, link adjacent screw members 210 and 230 by a link member, e.g., 250, 450 and 550, provides a larger bearing surface to support or carry the load from the femur head than conventional pinning screws. The load on a hip is typically downward. Therefore, the additional bearing surface created by a bone fixation assembly constructed in accordance with one or more aspects of the present invention transfers the load on the hip down to the screw members and link member of the bone fixation assembly.

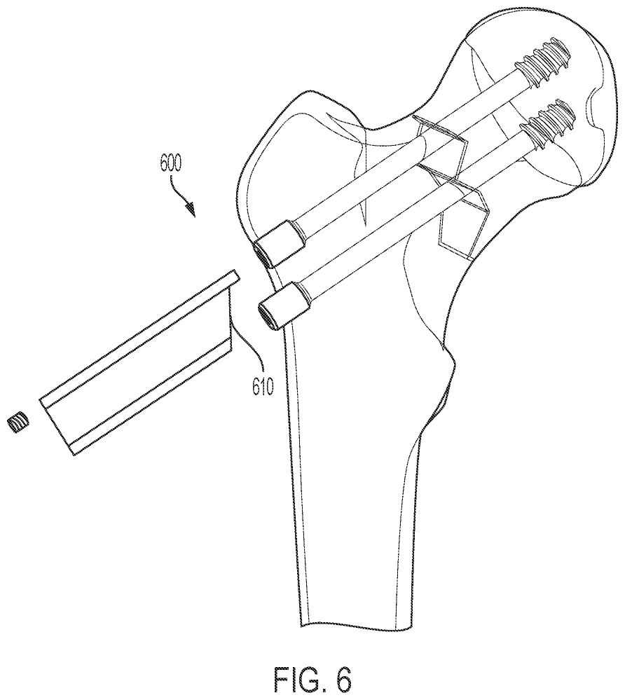

FIG. 6 depicts another embodiment of a plate member 600 having an angled end 610. In this example, screw members 210, 230 are inserted in a vertical configuration into bone and angled end 610 accommodates the vertical arrangement and configuration of screw members 210, 230.

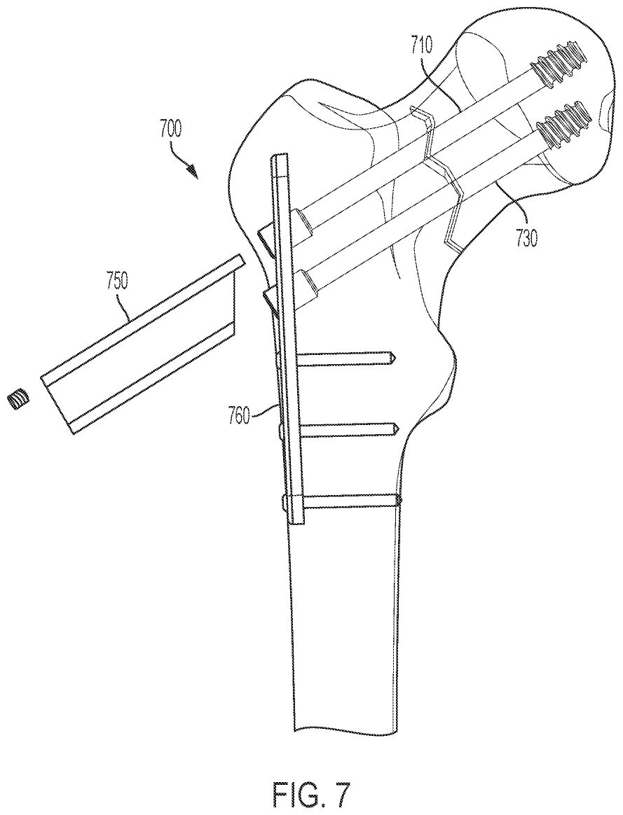

FIG. 7 depicts another example of bone fixation assembly 700 constructed in accordance with one or more aspects of the present invention that may be utilized with a bone plate 760 extending vertically along the femur. In this example, screw members 710 and 730 and link member 750 pass through apertures (e.g. holes or slots) formed in bone plate 760.

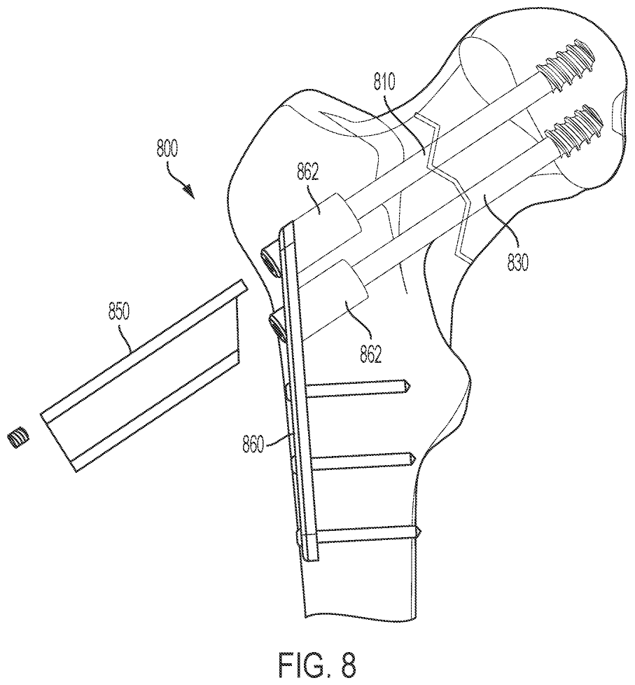

FIG. 8 depicts another example of bone fixation assembly 800 constructed in accordance with one or more aspects of the present invention that may be utilized with a bone plate 860 extending vertically along the femur. In this example, screw members 810 and 830 pass through barrels or sleeves 862 extending from bone plate 860. Barrels or sleeves 862 and bone plate 860 may also be configured to slidably receive plate member 850 for insertion and receipt by screw members 810, 830 as described above.

Bone fixation assemblies 700 and 800 constructed in accordance with one or more aspects of the present invention are dynamic and may prevent screw members from sliding relative to a bone plate.

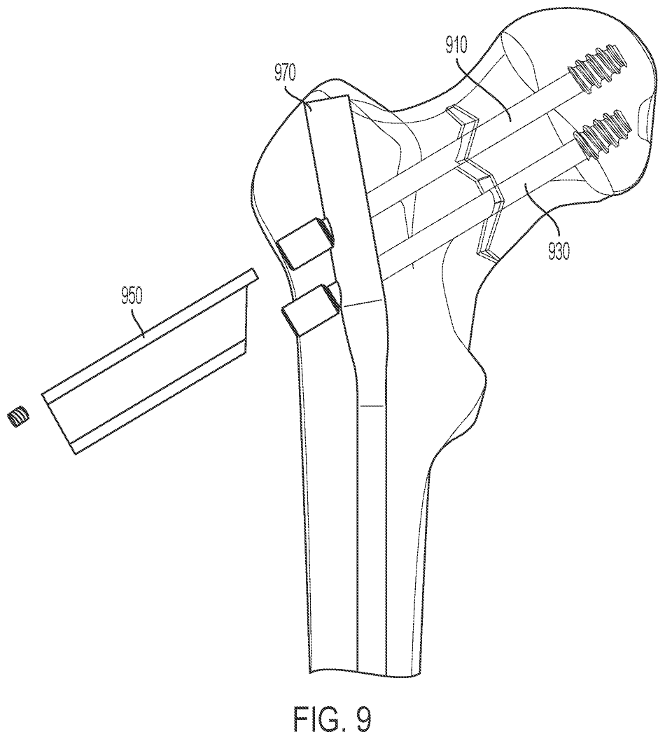

FIG. 9 depicts another example of a bone fixation assembly 900 constructed in accordance with one or more aspects of the present invention that may be utilized with an IM nail 970. In this example, bone screws 910 and 930 and link member 950 pass through an upper portion of IM nail 970.

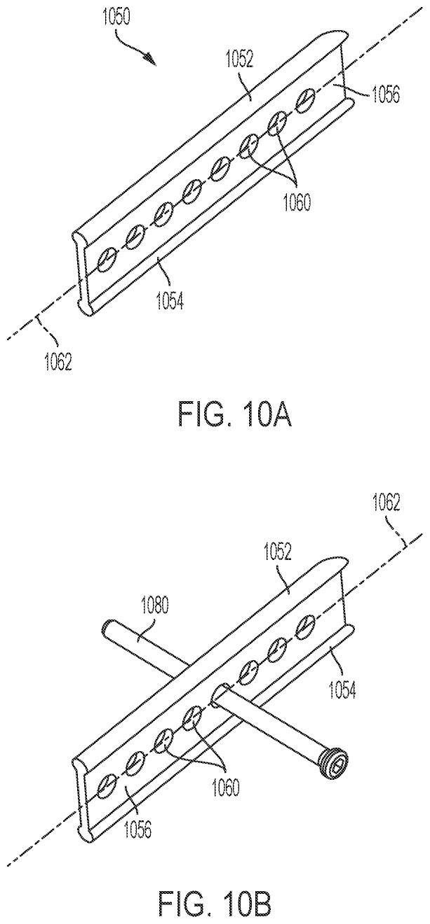

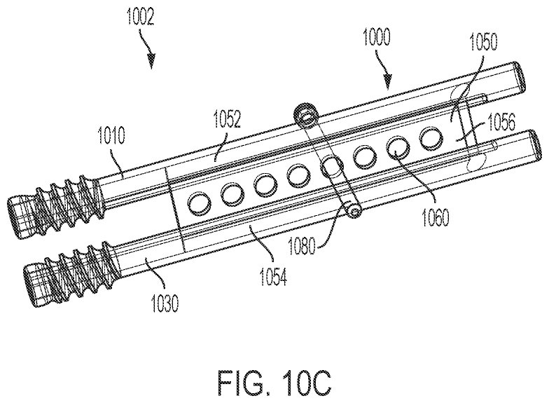

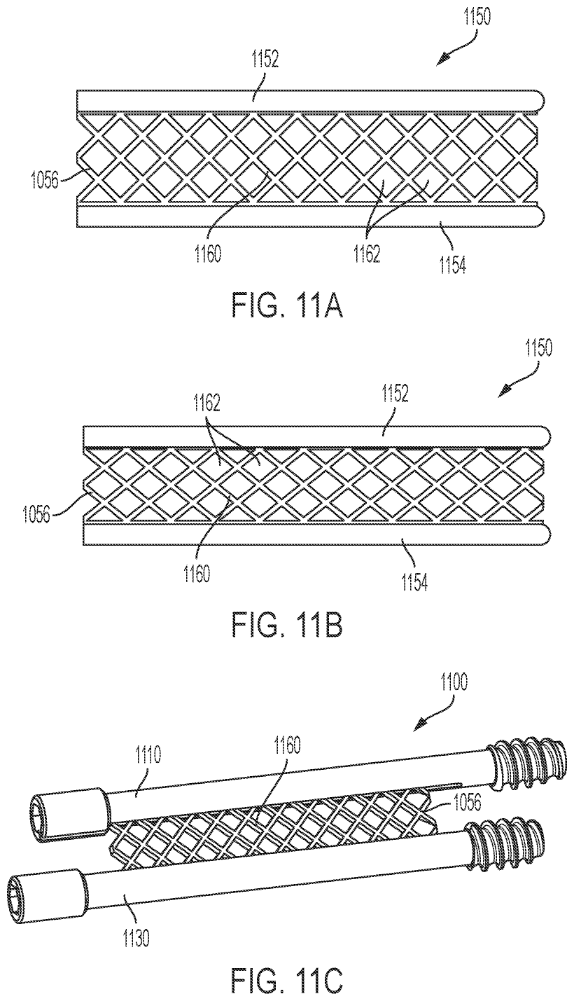

In an alternative embodiment depicted in FIGS. 10A-10C, plate member 1056 of link member 1050 may include one or more apertures 1060 extending between first lateral side 1052 and second lateral side 1054. As illustrated in FIG. 10A, plate member 1056 may include a plurality of apertures 1060 spaced apart from each other and along a longitudinal axis 1062 of plate member 1056. As illustrated in FIGS. 10B and 10C, apertures 1060 may be sized, shaped and configured to receive a third screw member 1080 to provide cross locking capability of bone fixation assembly 1000. In this example, third screw member 1080 is inserted perpendicular or, alternatively, at an angle relative to plate member 1056 through one of apertures 1060. Third screw member 1080 may be any type of screw member or nail configuration known in the art. The unused apertures 1060 may, for example, allow for more blood flow providing vascular access as the bone fracture heals. As shown, for example, in FIG. 10C, link member 1050 may be inserted between screw member 1010 and screw member 1030 in, for example, the same manner as described above and illustrated in FIG. 11D. Bone fixation assembly 1000 may then be installed or implanted into a calcaneous bone 1002 while third screw member 1080 provides cross locking of the bone fixation assembly.

In an alternative embodiment depicted in FIGS. 11A-11C, plate member 1156 of link member 1150 may include a flexible and/or compressible structure 1160 extending between first lateral side 1152 and second lateral side 1154. As illustrated in FIG. 11A, flexible and/or compressible structure 1160 may be configured to include a two-dimensional or three-dimensional lattice structure including a plurality of apertures 1162. In operation, lattice structure 1160 is configured and capable of being compressed or expanded in order to provide motion to, for example, bring screw members 1110 and 1130 together to provide residual compressive forces on fracture faces between screw members 1110 and 1130, activating the mechanostat osteo inductive cascade. If lattice structure 1160 provides motion to separate screw members 1110 and 1130, it could be useful to, for example, counteract subsidence in certain indication with loads or bone quality such that subsidence would result in shortened or malreduction. Apertures 1162 may, for example, allow for more blood flow providing vascular access as the bone fracture heals. Link member 1150 may be inserted between screw member 1110 and screw member 1130 in, for example, the same manner as described above and illustrated in FIG. 11C. As shown, for example, in FIG. 11D, bone fixation assembly 1100 may be installed or implanted into a calcaneous bone 1102.

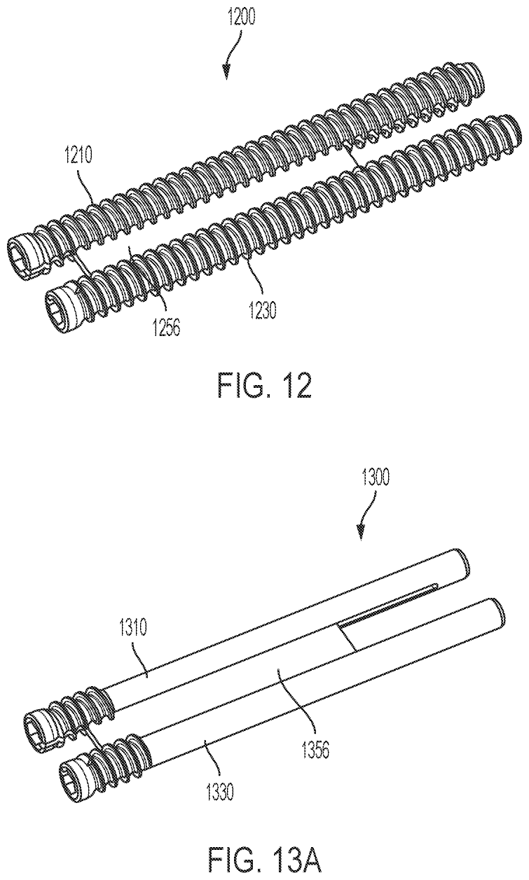

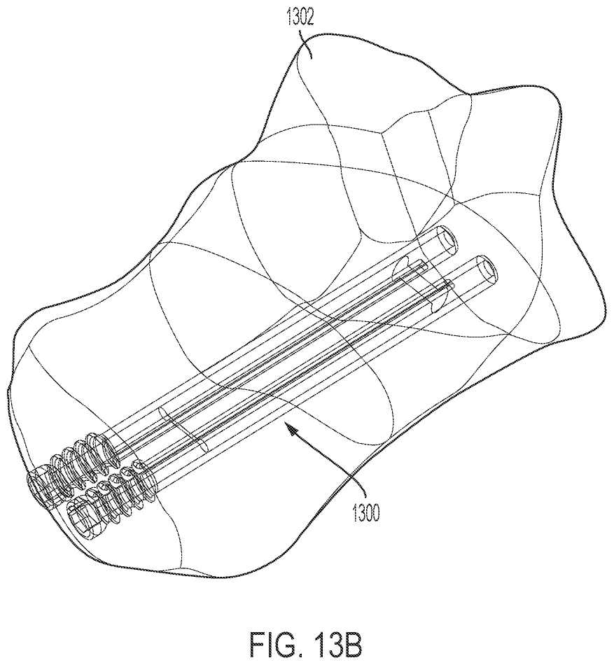

FIG. 12 illustrates an alternative embodiment of bone fixation assembly 1200 constructed in accordance with one or more aspects of the present invention. In this example, screw members 1210 and 1230 may include threads that extend from a proximal end to a distal end. FIG. 13 illustrates yet another embodiment of bone fixation assembly 1300 with screw members 1310 and 1330 being, for example, trailing thread screw members having threads only at a proximal end of its shaft. In the examples illustrated in FIGS. 12 and 13, link members 1250 and 1350 include solid plate members 1256 and 1356. However, alternative plate members may be used that include, for example, one or more apertures or two- or three-dimensional lattice structures that may or may not compress or expand. Bone fixation assemblies 1200 and/or 1300 may be inserted or implanted into a calcaneous bone 1302 (as illustrated in FIG. 13B).

In one embodiment, a bone fixation assembly 200, for example, constructed in accordance with one or more aspects of the present invention may be installed or implanted using, for example, a targeting or installation guide instrument constructed to appropriately place screw members 210, 230 in the correct orientation and spacing for slidably receiving link member 250. Once screw members 210, 230 are installed, using techniques known in the art, first and second lateral sides 252, 254 and plate member 256 of link member 250 may be slide through openings 217 and into slots 226 and channels 229. In one example, link member 250 may be hammered into place because of the porosity of the bone. Once link member 250 is fully installed within screw members 210, 230, locking screw 490 may be installed to retain lateral sides 252 and 254 within channels 229 of screw members 210, 230. Once installed or implanted into the bone, bone fixation assembly 200 provides an anti-rotation and anti-subsidence solution to, for example, fix a hip fracture.

For any bone fixation assembly constructed in accordance with one or more aspects of the present invention, the screw members and the link member extending between the screw members may be many different sizes, shapes and configurations. The embodiments described and illustrated showing the screw members as having, for example, thread on a leading edge (e.g. 210, 230), threads on a trailing edge (e.g. 1310, 1330) or threads extending the length of the screw member (e.g. 1210, 1230) are only some examples of the sizes, shapes and configuration that may be used. Also, the embodiments described and illustrated showing the link members as having, for example, a sold plate member (e.g. 256), a plurality of apertures (e.g. 456, 1056) or a two- or three dimensional structure (e.g. 1056) are only some examples of the sizes, shapes and configuration that may be used.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A bone fixation assembly, said bone fixation assembly comprising:
a first screw member, said first screw member including a shaft extending along a longitudinal axis, the shaft including a proximal end and a distal end, the shaft including an axial surface extending between the proximal end and the distal end, the shaft including a slot in the axial surface extending from the proximal end along the longitudinal axis;
a second screw member, said second screw member including a shaft extending along a longitudinal axis, the shaft including a proximal end and a distal end, the shaft including an axial surface extending between the proximal end and the distal end, the shaft including a slot in the axial surface extending from the proximal end along the longitudinal axis; and
a link member, said link member including a first lateral side, a second lateral side and a plate member extending between the first lateral side and the second lateral side, the first lateral side being slidably received by the slot in the axial surface of the shaft of said first screw member, and the second lateral side being slidably received by the slot in the axial surface of the shaft of said second screw member.

2. The bone fixation assembly of claim 1, wherein the plate member includes a plurality of apertures.

3. The bone fixation assembly of claim 1, wherein the plate member includes a lattice structure.

4. The bone fixation assembly of claim 3, wherein the lattice structure is flexible.

5. The bone fixation assembly of claim 3, wherein the lattice structure is configured to compress or expand.

6. The bone fixation assembly of claim 1, further comprising a third screw member, said third screw member passing through the plate member to provide cross locking of said bone fixation assembly.

7. The bone fixation assembly of claim 1, wherein the first screw member includes threads extending along at least a portion of the shaft.

8. The bone fixation assembly of claim 7, wherein the threads are at the proximal end of the shaft.

9. The bone fixation assembly of claim 7, wherein the threads are at the distal end of the shaft.

10. The bone fixation assembly of claim 7, wherein the threads extend from the proximal end to the distal end of the shaft.

11. The bone fixation assembly of claim 1, further comprising a bone plate, said first screw member, said second screw member and said link member passing through said bone plate, wherein the proximal ends of said first and second screw members couple to said bone plate.

12. The bone fixation assembly of claim 1, further comprising a bone nail, said first screw member, said second screw member and said link member passing through said bone nail, wherein the proximal ends of said first and second screw members couple to said bone nail.

13. A bone fixation assembly, said bone fixation assembly comprising:
a first member, said first member including a shaft extending along a longitudinal axis, the shaft including a proximal end and a distal end, the shaft including an axial surface extending between the proximal end and the distal end, the shaft including a slot in the axial surface extending from the proximal end along the longitudinal axis;
a second member, said second member including a shaft extending along a longitudinal axis, the shaft including a proximal end and a distal end, the shaft including an axial surface extending between the proximal end and the distal end, the shaft including a slot in the axial surface extending from the proximal end along the longitudinal axis; and
a link member, said link member including a first lateral side, a second lateral side and a plate member extending between the first lateral side and the second lateral side, the first lateral side being slidably received by the slot in the axial surface of the shaft of said first member, and the second lateral side being slidably received by the slot in the axial surface of the shaft of said second member.

14. The bone fixation assembly of claim 13, wherein said bone fixation assembly is configured to be installed in a hip joint.

15. The bone fixation assembly of claim 13, wherein said bone fixation assembly is configured to be installed in a calcaneous bone.

16. The bone fixation assembly of claim 13, wherein said first member includes threads on the axial surface at the proximal end.

17. The bone fixation assembly of claim 13, wherein said second member includes threads on the axial surface at the distal end.

18. The bone fixation assembly of claim 13, wherein said link members includes a plurality of apertures formed in the plate member.

19. The bone fixation assembly of claim 13, wherein the plate member is flexible.
